(19) United States
(12) Patent Application Publication
(10) Pub. No.: US 2024/0091438 A1
(43) Pub. Date: Mar. 21, 2024

(54) System to Provide Multiple Infusions to a Patient

(71) Applicant: FRESENIUS VIAL SAS, Brézins (FR)

(72) Inventors: Maarten Nijsten, Groningen (NL); Emeric Mermet, Saint Martin Le Vinoux (FR); Frank Doesburg, Zuidlaren (NL)

(21) Appl. No.: 18/255,204

(22) PCT Filed: Dec. 21, 2021
(86) PCT No.: PCT/EP2021/087022

§ 371 Date: May 31, 2023

(30) Foreign Application Priority Data
Dec. 28, 2020 (EP) 20315508.0
Mar. 30, 2021 (EP) 21165897.6

(51) Int. Cl. A61M 5/172; A61M 5/14; A61M 5/168; G16H 20/17; G16H 40/63

(52) U.S. Cl. CPC A61M 5/172; A61M 5/1407; A61M 5/16827; G16H 20/17; G16H 40/63; A61M 2202/0007; A61M 2205/33; A61M 2205/505

(57) ABSTRACT

The invention relates to a system for managing multiple infusions to a patient, comprising a control device (2) configured to control a multiplicity of infusion devices (10) for administering a multiplicity of medical fluids through at least one infusion line (100) of an infusion set to the patient (P), the multiplicity of medical fluids to the multiplicity of infusion devices (10) being assigned, preferably by a user characterized in that: —the control device (2) determines compatibility and non-compatibility of the assigned multiplicity of medical fluids, —In that the control device (2) displays to a user compatibility information regarding the compatibility or non-compatibility of the multiplicity of medical fluids, —and in that at least one of the multiplicity of medical fluids to the at least one infusion line is assigned, preferably by a user, in accordance with the compatibility information.

The invention relates to a system for providing multiple infusions to a patient. The invention is also related to a Human-Machine Interface (HMI) to be used in conjunction with said system.

A system of this kind comprises a multiplicity of infusion devices for administering a multiplicity of medical fluids through infusion lines of an infusion set of the patient, and a control device for controlling the multiplicity of infusion devices.

Intravenous (IV) therapy is one of the most common forms of treatment in hospitals worldwide. Using an infusion pump, medical fluids or solutions such as drug solutions are delivered into the bloodstream of a patient at a preprogrammed rate. In many places where complex care is provided, such as intensive care units (ICUs), patients typically receive IV treatment using multiple infusion pumps. As many drugs are chemically incompatible, drugs are often administered through separate infusion channels (lumens) in order to avoid precipitation or inactivation.

The management of such compatibilities at the pump level, usually manually checked and performed by a nurse at the bedside, is complex, cumbersome and time consuming. Such pumps may be complex to operate individually, and within a multi-infusion therapy their use must be aligned and harmonized, which is prone to human error and therefore comes with a risk of medication errors. The multitasking involved with the simultaneous operation of multiple infusion pumps also increases the workload of an ICU nurse and thereby likelihood of errors.

There hence is a need to improve the administration of multiple infusions to a patient in a more reliable and guided fashion such that risks for errors and strain on the user is lowered.

WO2019001879 discloses a system for providing multiple infusions to a patient, comprising a control device for controlling a multiplicity of infusion devices. This system requires a multiplex module configured to schedule packets of medical fluid out of the multiplicity of medical fluids and to arrange the packets in a sequence for administration of the medical fluids of the packets.

The main idea of WO2019001879 is to perform repetitive sequences of given drugs, the compatibility or non-compatibility being dealt with thanks to external databases and dedicated computational abilities which are able to infuse the correct amount of medication in accordance with the prescribed medication. The content of WO2019001879 is incorporated herein by reference.

Nevertheless, because the actual infused fluids are calculated and dealt with based on the computational abilities of the provided system, this may cause some limitations on the implementation of the system in the current healthcare organizations.

A first limitation may be that the responsibility of the infusion is relying on an automated system, which is less nurse-dependent. This leads to higher responsibilities of the infusion system, which may not be acceptable from the hospital standpoints.

A second limitation may come from the repetitive infusion sequences which encompass different actual flow rate—at a given time—than the one prescribed by the practitioner. This system could require additional and long validation before authorizing such innovative infusion sequences. Indeed, the effects and efficiency of such sequences and mixed drugs need to be investigated by medical authorities as long as the sequential use of incompatible drugs within the same lumen.

An alternative technique is presented in US2014067425 which discloses the generation of an infusion map system illustrating routes of administration. Compatibility of a new order is checked and displayed to determine if the new order is compatible with the existing infusion map. A recommended configuration is highlighted to the user who can accept or modify the suggested configuration.

Nevertheless, this solution does not provide with a list of compatible drugs and routes and then can lead to a cumbersome check if several drugs are to be administered with a potential high number of incompatibilities. Moreover, the displayed routes are not intuitively shown since all infusion routes are presented one aside the others. The US2014067425 document is also silent to teach how to manipulate the drug compatibility information and to navigate into it.

Similar documents, like WO16141012, WO05101279 and WO11109779 are directed to infusion systems and method which are also silent to address the afore mentioned issues.

It is an object of the invention to provide a dedicated system being able to deal with the compatibility and non-compatibility of drugs to be infused in a guided and safe operation, giving the user—typically the nurse—the possibility to handle this complex operation.

It is also an object of the invention to provide a dedicated HMI workflow, either at the pump level or through a remote pump controller, like a mobile device or a computer, to guide the nurse in the management of the complex preparation and operation of the infusions.

It is preferably also an object of the invention to provide the personnel in charge of the drug library, typically the pharmacist, with a dedicated system to address the compatibility of drugs or solutions to be infused.

More precisely, the object of the invention is to propose a system for managing multiple infusions to a patient, comprising a control device configured to control, a multiplicity of infusion devices for administering a multiplicity of medical fluids through at least one infusion line of an infusion set to the patient,

the multiplicity of medical fluids to the multiplicity of infusion devices being assigned, preferably by a user, characterized in that
the control device determines compatibility and non-compatibility of the assigned multiplicity of medical fluids,
in that the control device displays to a user compatibility information regarding the compatibility or non-compatibility of several said medical fluids, —and in that at least one of the multiplicity of medical fluids to the at least one infusion line is assigned, preferably by a user, in accordance with the compatibility information.

The medical fluids refer to drugs or solutions like lactated ringers, saline or dextrose, cited as non-limitative examples. In the following, the term “drug(s)” is selected as an example and the systems, described further on, also refer to any other infusible medical fluids.

The compatibility information is typically provided at the control device as a compatibility list which can be in one embodiment in the shape of a matrix of columns and lines, preferably wherein the list being editable and/or searchable by the user.

Preferably, upon the selection of one of the several infusible medical fluids, information on the compatibility, non-compatibility or even unknown compatibility of the selected medical fluid with at least one another of the several infusible fluids is provided at the control device to the user, based on the compatibility information.

Typically, upon selection of two incompatible medical fluids, the information of the non-compatibility is displayed at the control device to the user, based on the compatibility information.

In an additional embodiment, the control device is further configured to assist the user with a drug-to-lumen—or fluid-to-lumen assignation based on the compatibility information, preferably by providing guidance to address one or several medical fluids to one or several lumens for administration.

In another embodiment, the control device is further configured to display a schematic patient body shape with at least one of a localized single or a multiple-channel lumen entry, which is selectable by the user. In that example, upon selection of the one of a localized single or a multiple-channel lumen, a first fluid being already assigned to the one of a localized single or a multiple-channel lumen, and upon the selection of a second fluid to be infused, the control device may be further configured to provide information on compatibility or non-compatibility of the first and second fluids based on the compatibility information.

In an alternative embodiment, the invention is also characterized in that at least one association of an infusing device and an assigned fluid to the infusing device is displayed.

For instance, the at least one association is displayed as a row or a progress bar, also referred to as a swim lane. Preferably, the control device is further configured to provide the user with an updated view of the at least one association with a corresponding lumen indicator.

The lumen indicator may be a number and/or a dedicated color and/or a color scheme or pattern depending on the selected lumen. It can also be an actual intelligible text.

The control device may comprise a touchscreen in a wired or wireless connection to the multiplicity of infusion devices, the touchscreen commanding the infusion of the multiplicity of infusion devices. The control device can be a dedicated interface, a computer or a mobile device, like a smartphone or a tablet.

The invention is further related to a method for managing multiple infusions to a patient by controlling, using a control device, a multiplicity of infusion devices for administering a multiplicity of medical fluids through at least one infusion line of an infusion set to the patient, the method comprising:
assigning, preferably by a user, the multiplicity of medical fluids to the multiplicity of infusion devices,
determining, by the control device, compatibility and non-compatibility of the assigned multiplicity of medical fluids,
displaying to a user, at the control device, compatibility information regarding the compatibility or non-compatibility of the multiplicity of medical fluids,
assigning, preferably by a user, at least one of the multiplicity of medical fluids to the at least one infusion line in accordance with the compatibility information.

The idea underlying the invention shall subsequently be described in more detail with reference to the embodiments shown in the figures. Herein:

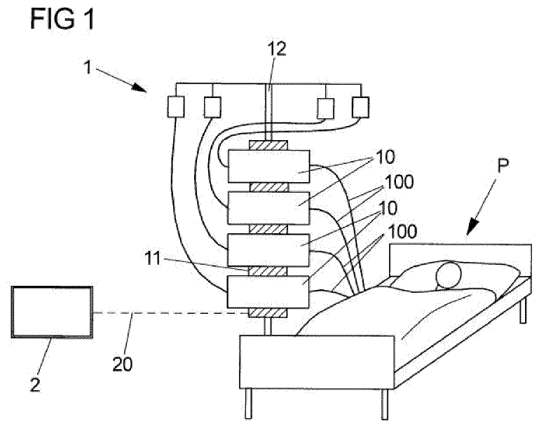

FIG. 1 shows a schematic drawing of an arrangement of multiple infusion set at a bedside;

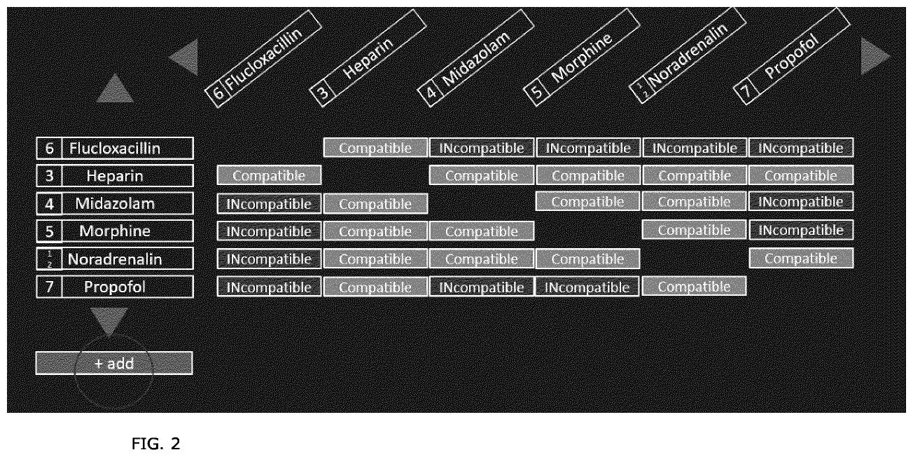

FIG. 2 shows a compatibility chart used in the present invention;

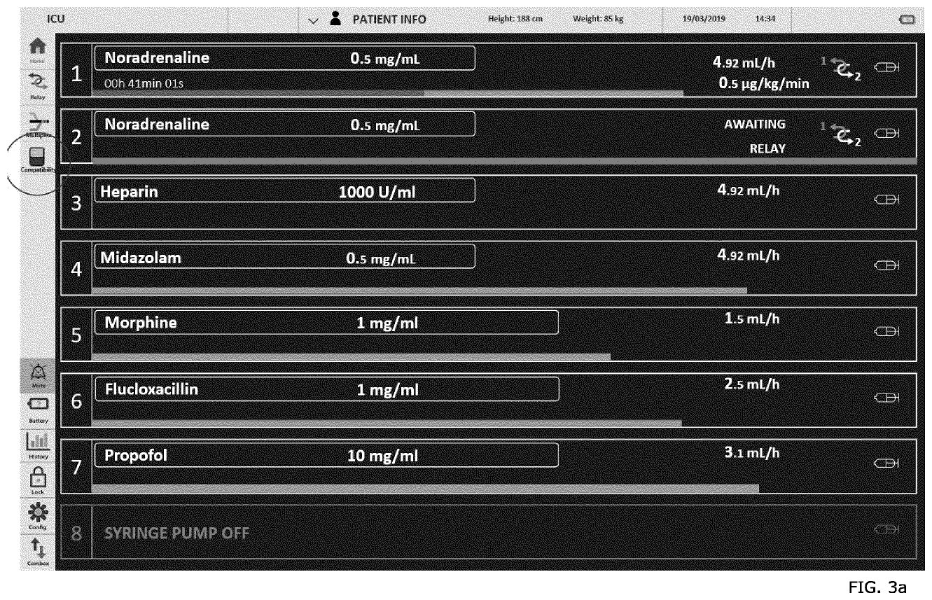

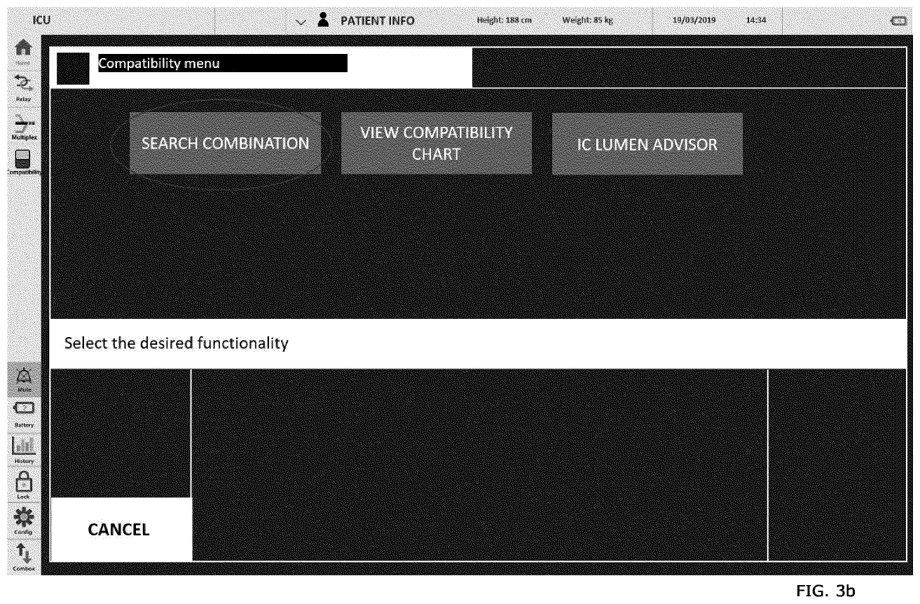

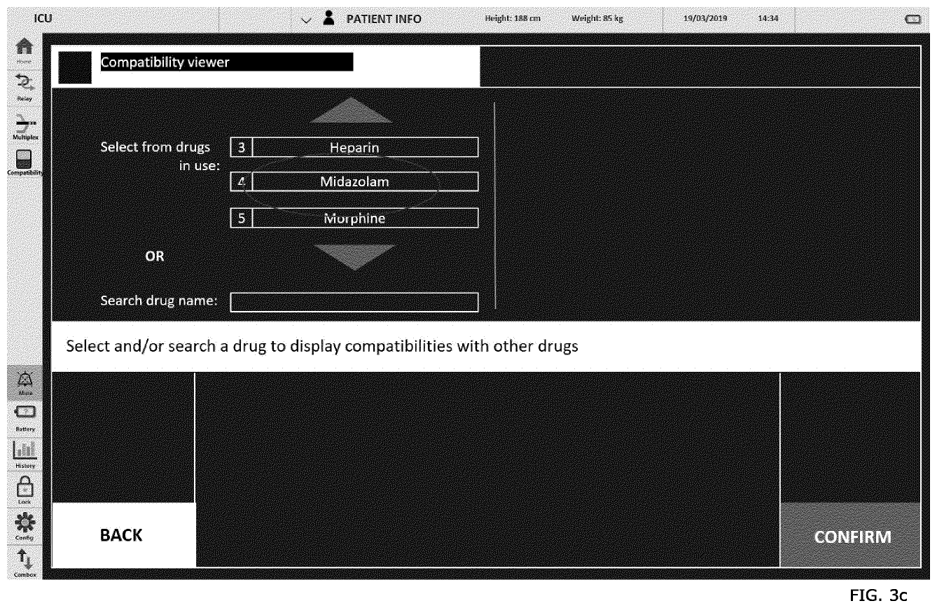

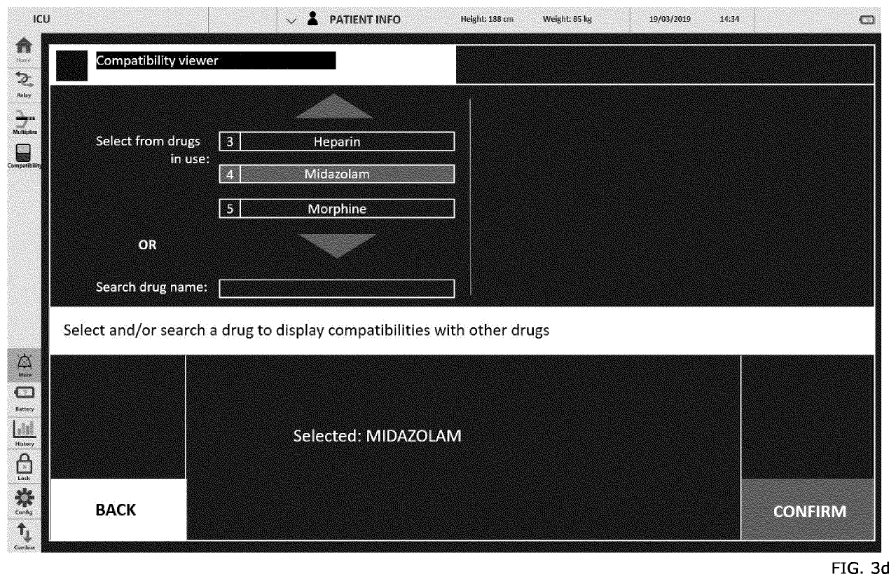

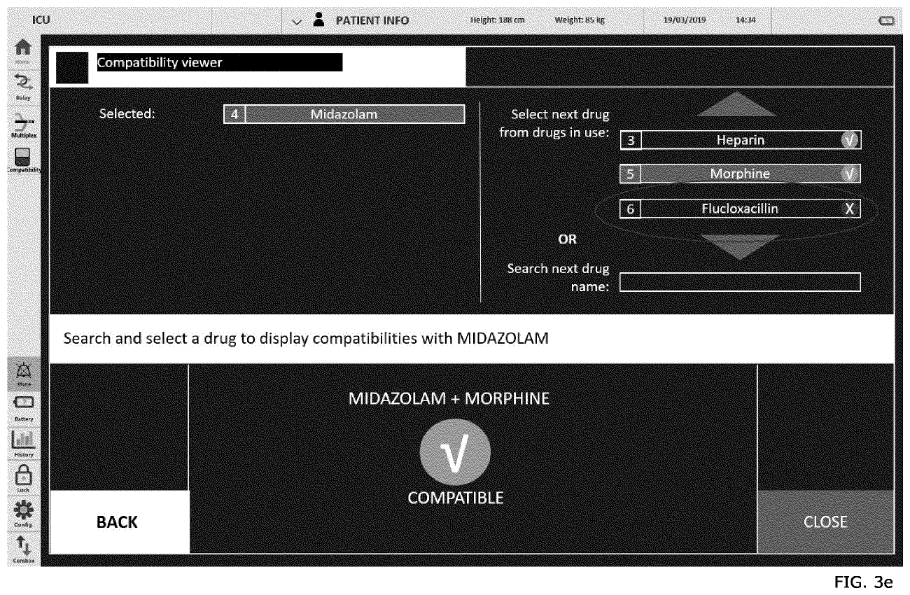

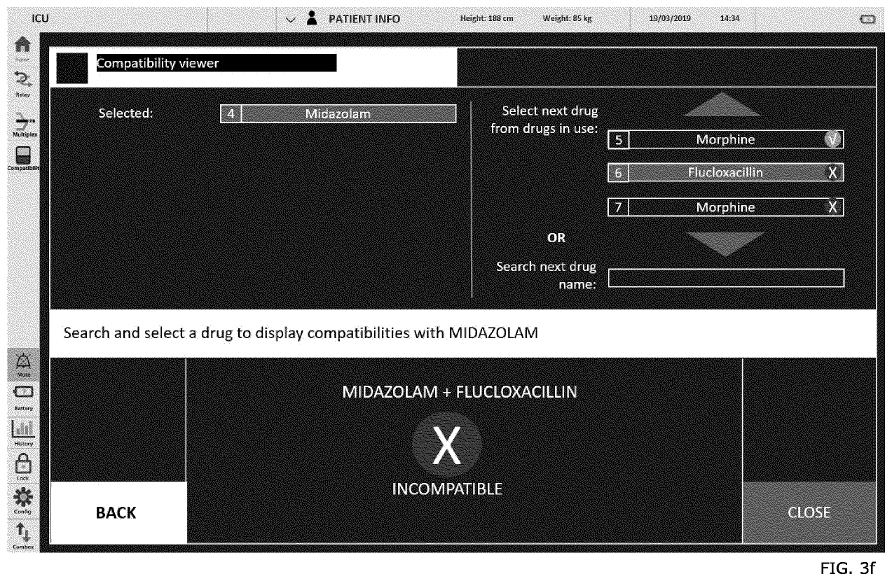

FIGS. 3a to 3f show a first workflow of operation related to a searching compatibility overview;

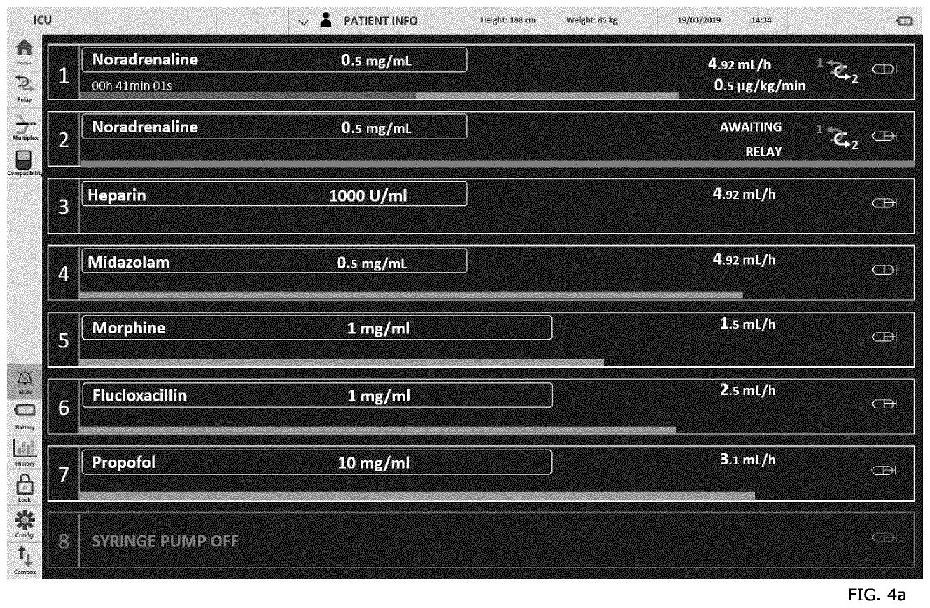

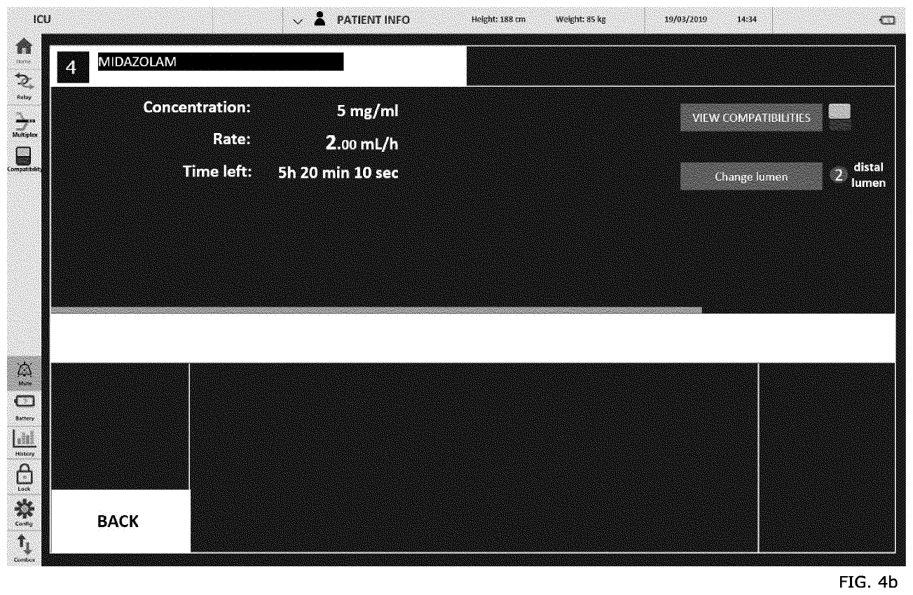

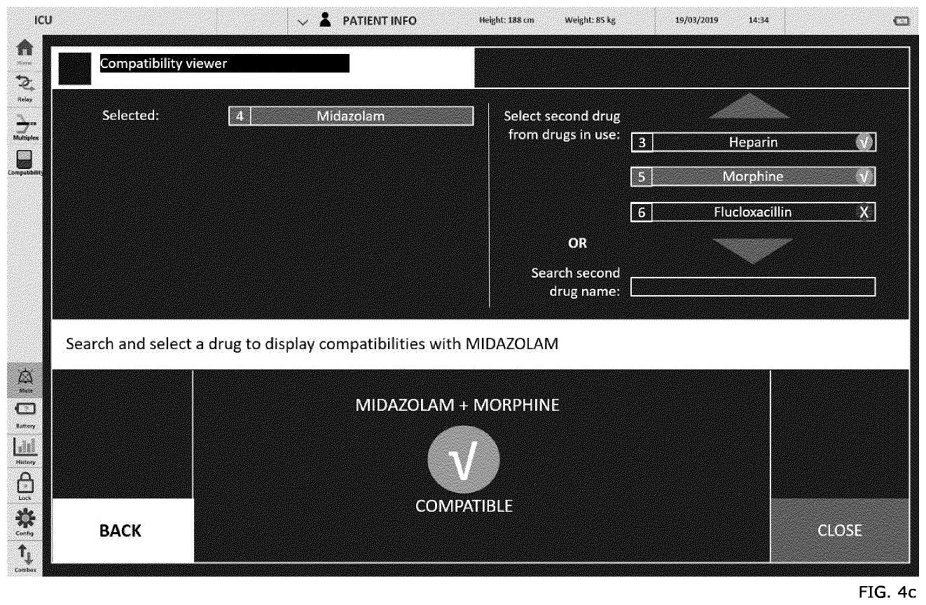

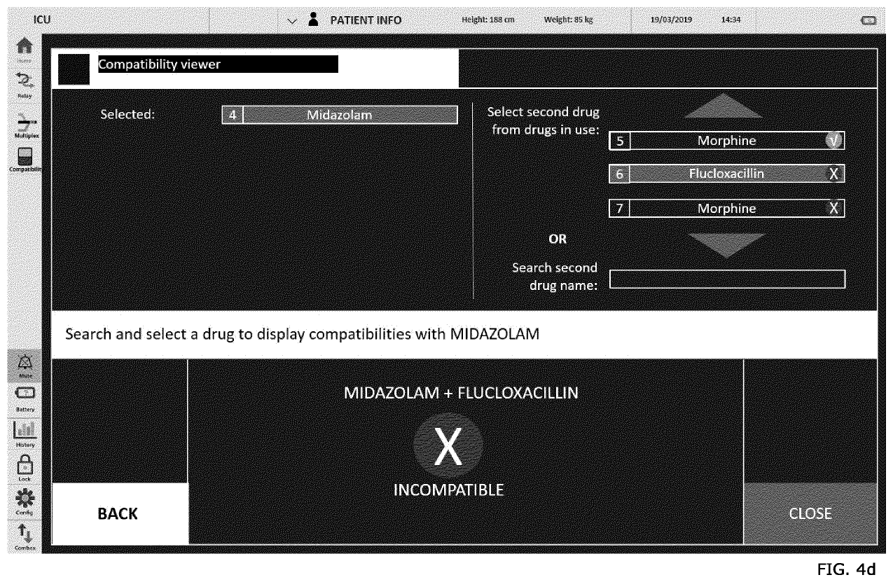

FIGS. 4a to 4d show a second workflow of operation related to searching compatibility from pump detail menu;

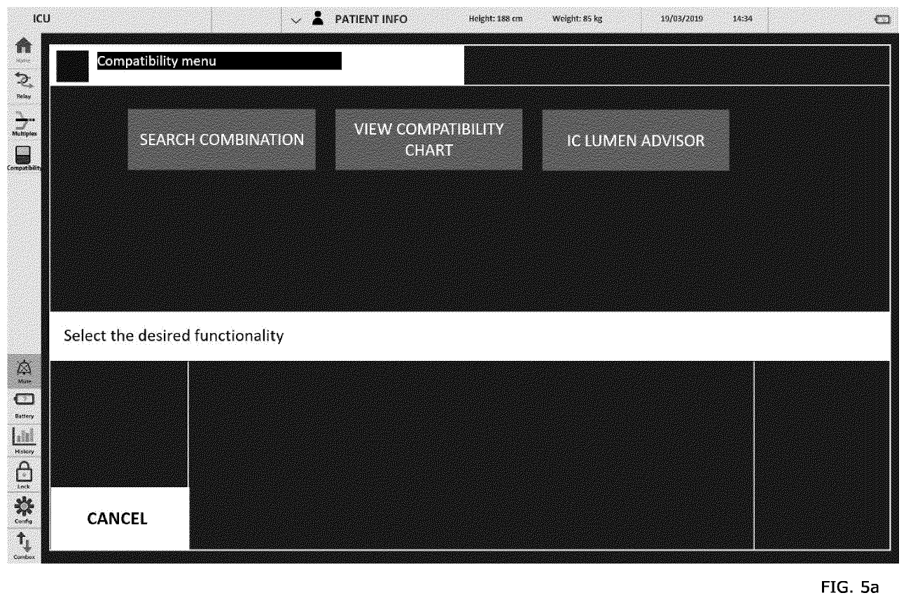

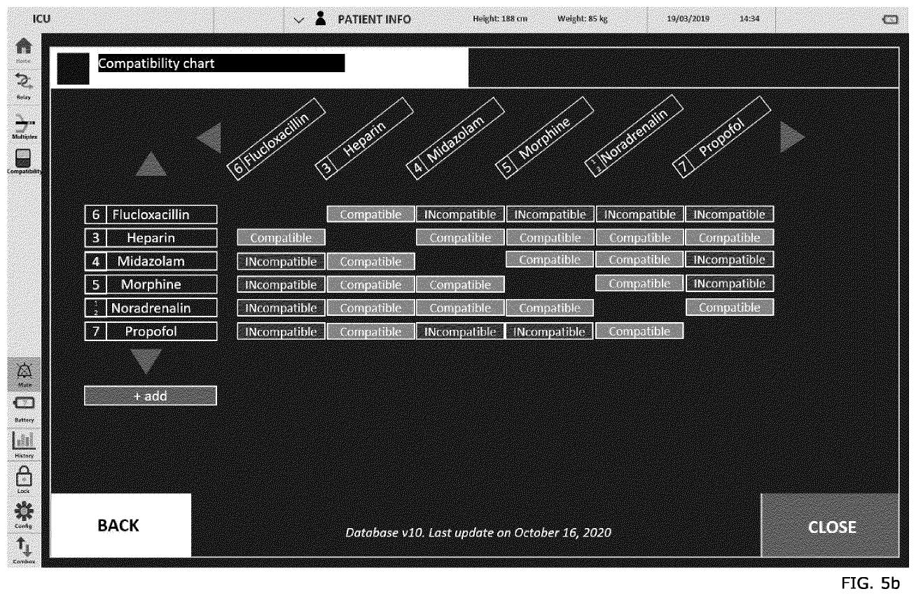

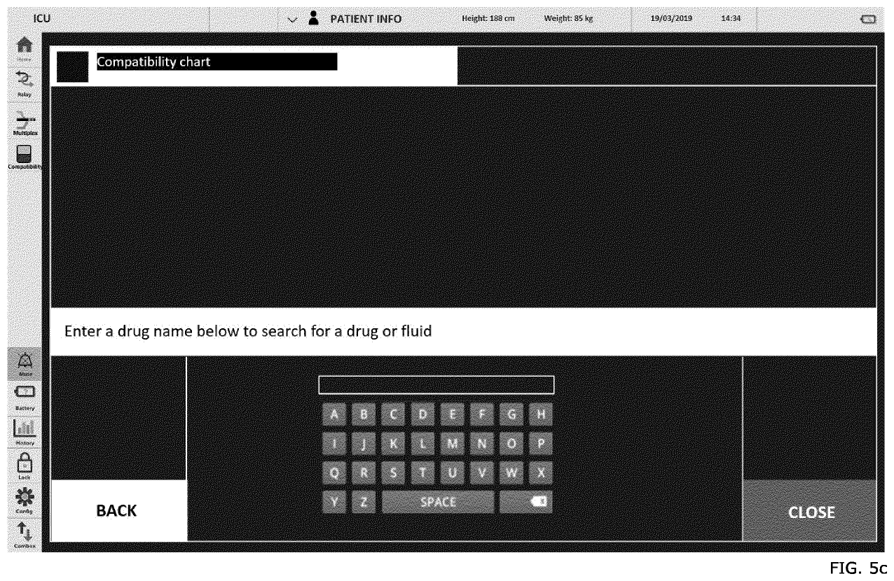

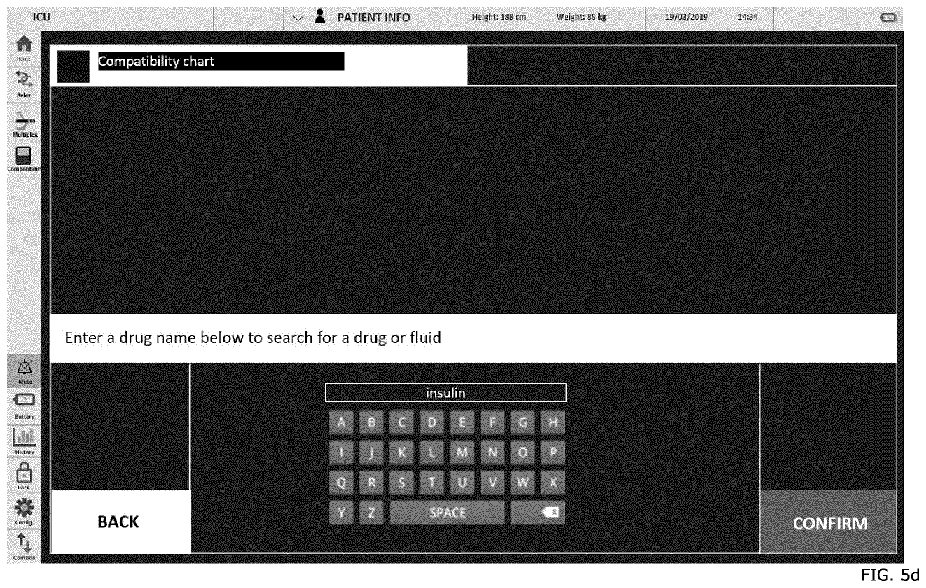

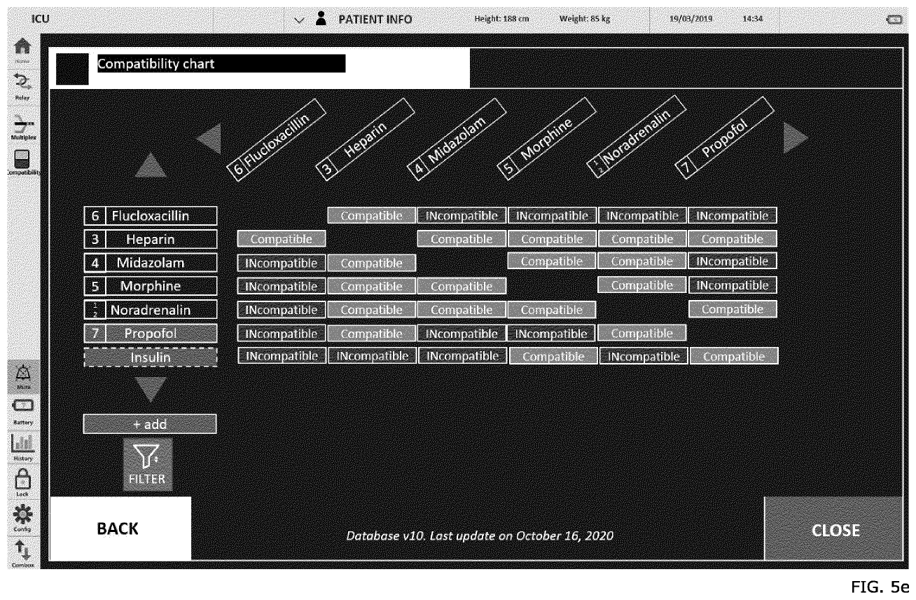

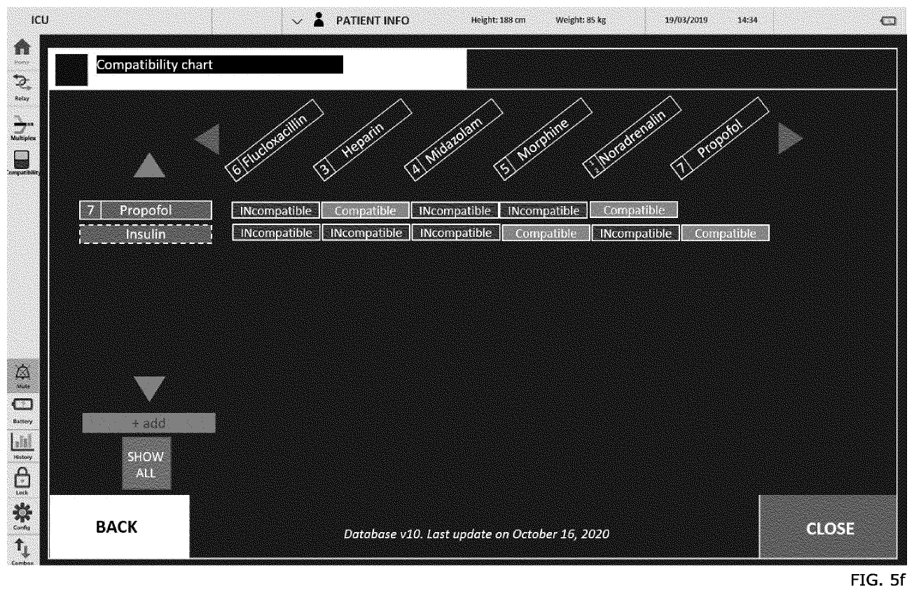

FIGS. 5a to 5f show a third workflow of operation related to the display of the compatibility chart and its selection;

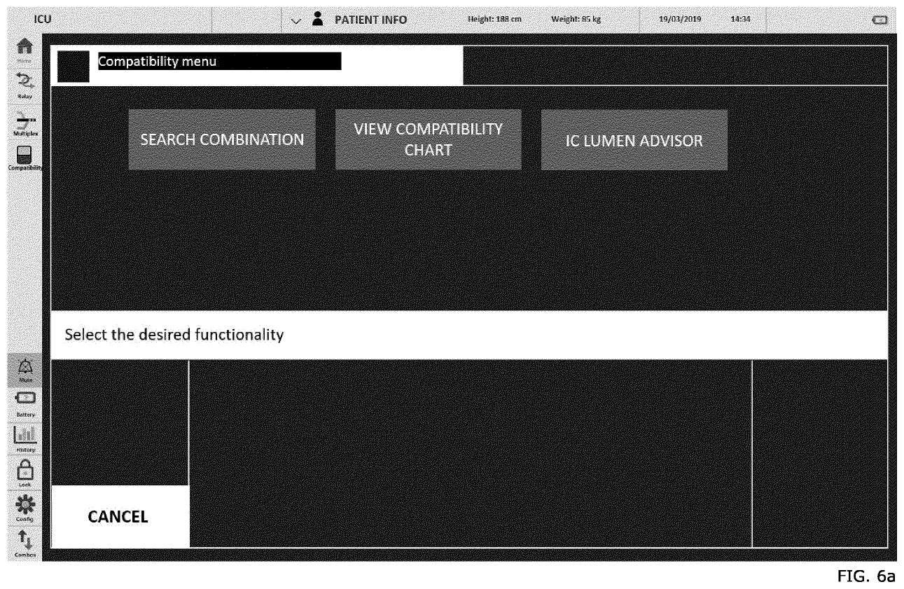

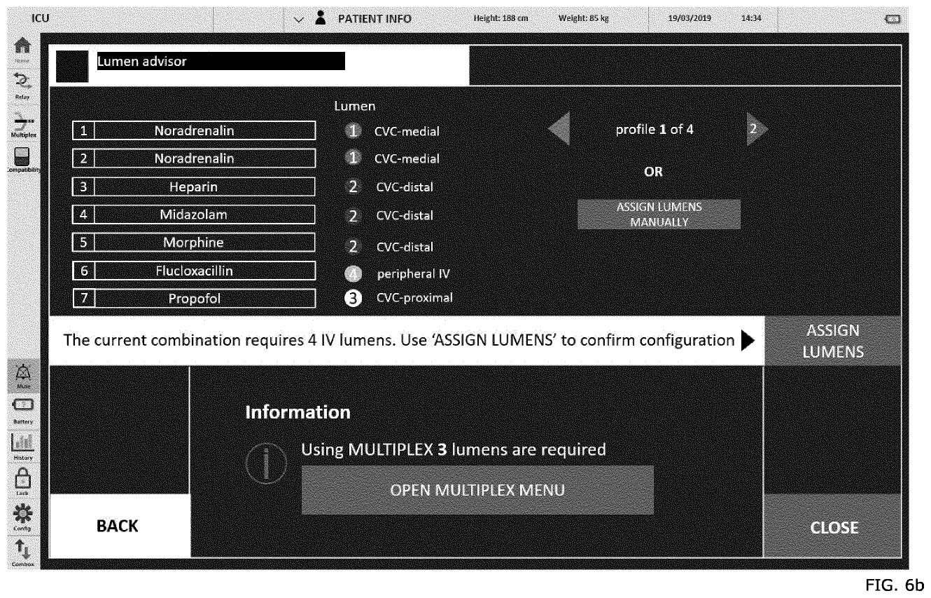

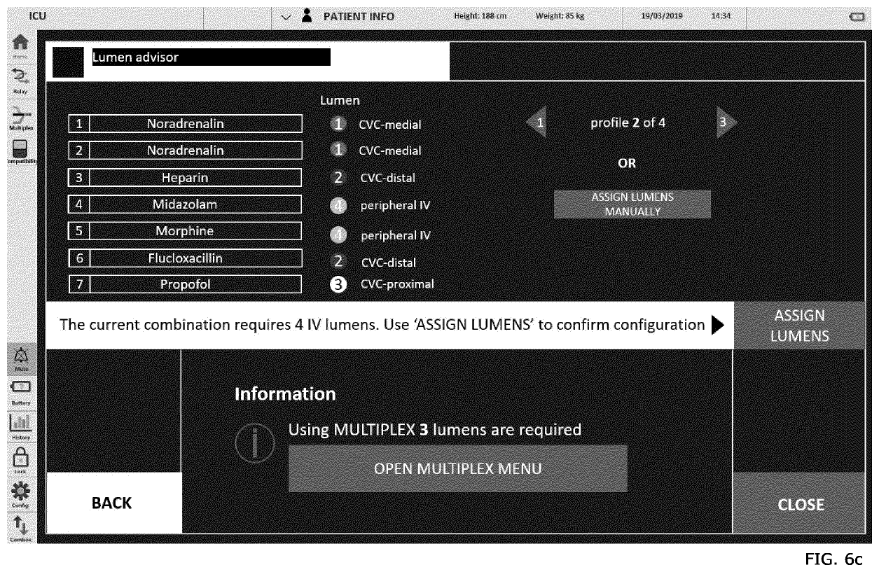

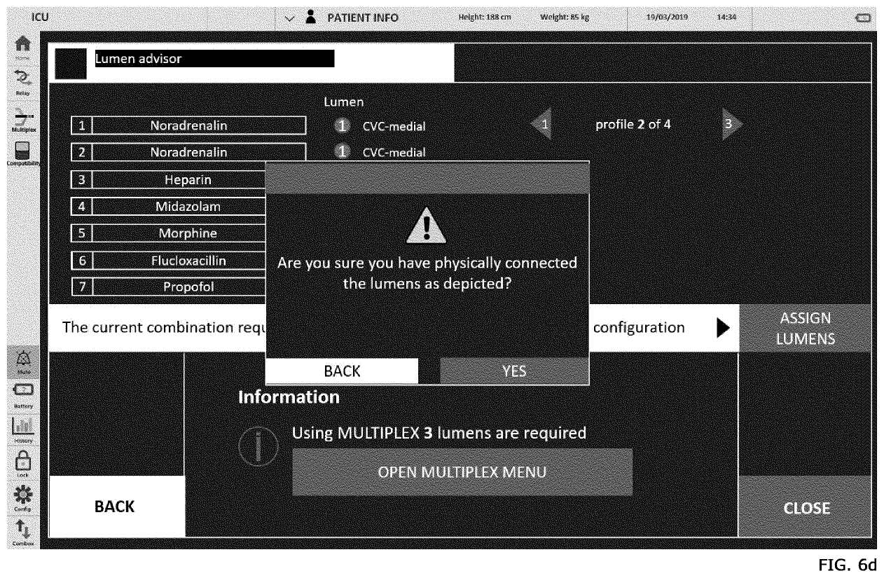

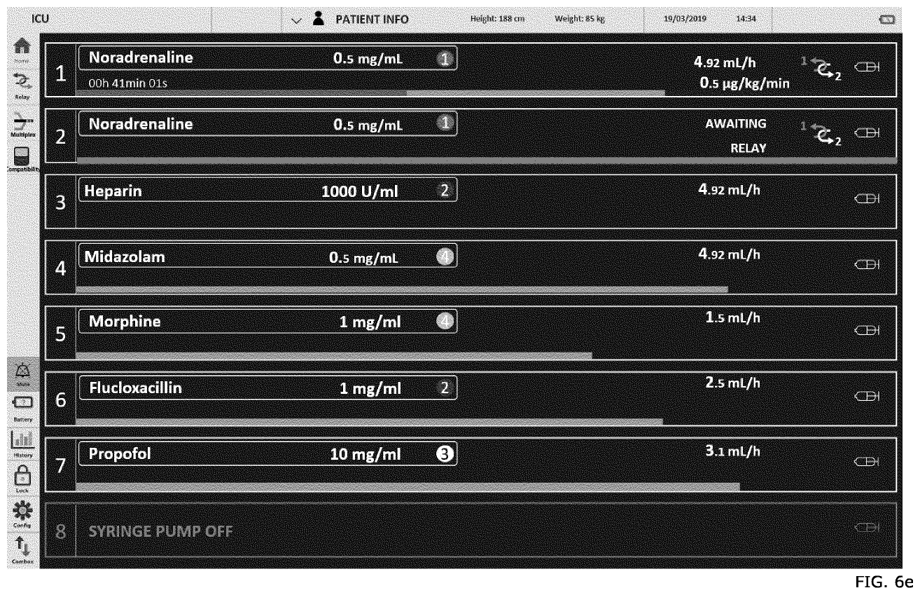

FIGS. 6a to 6e show a fourth workflow of operation related to the display of the compatibility chart and its selection;

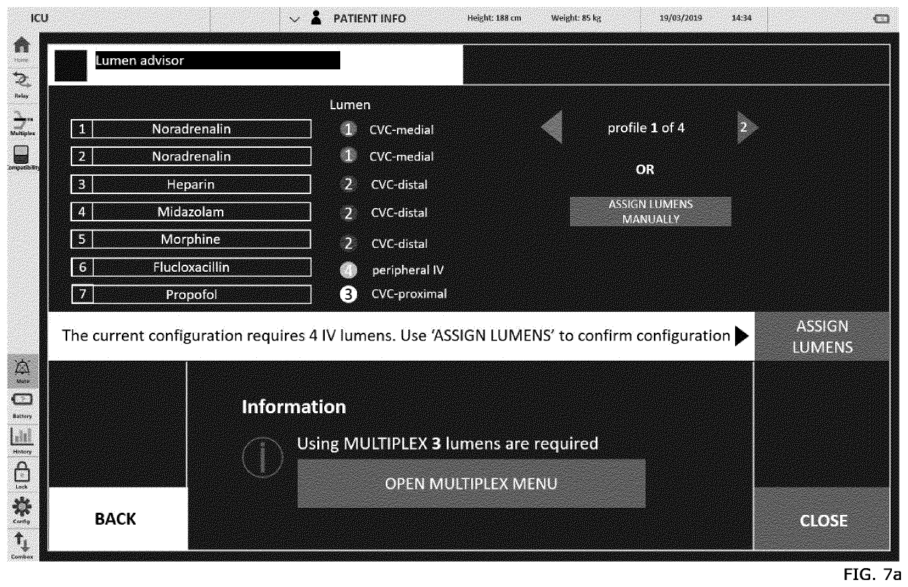

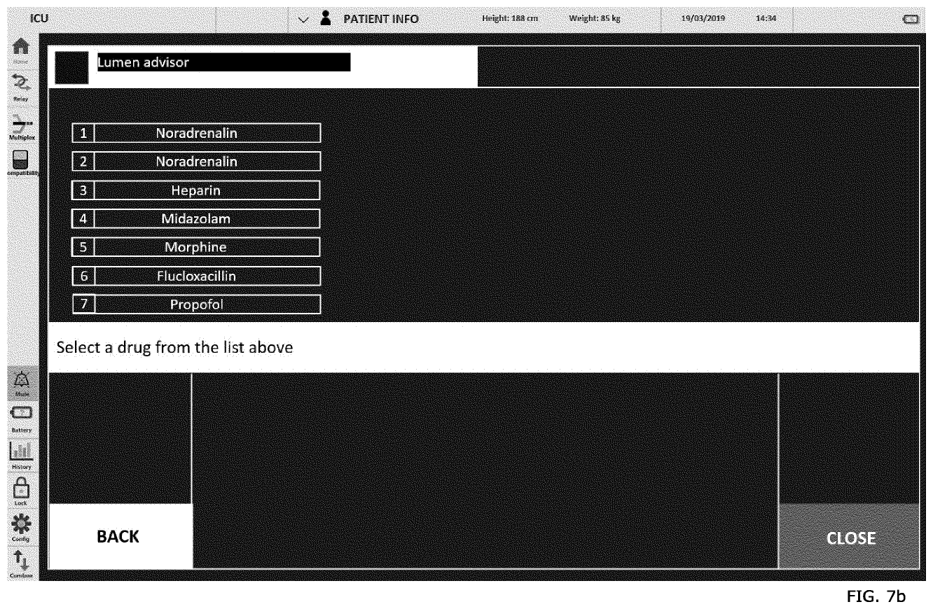

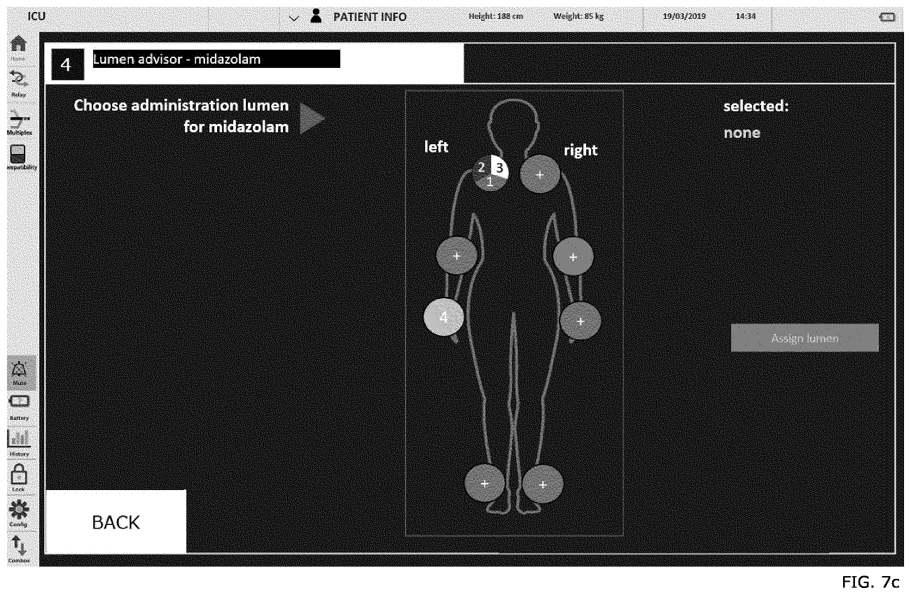

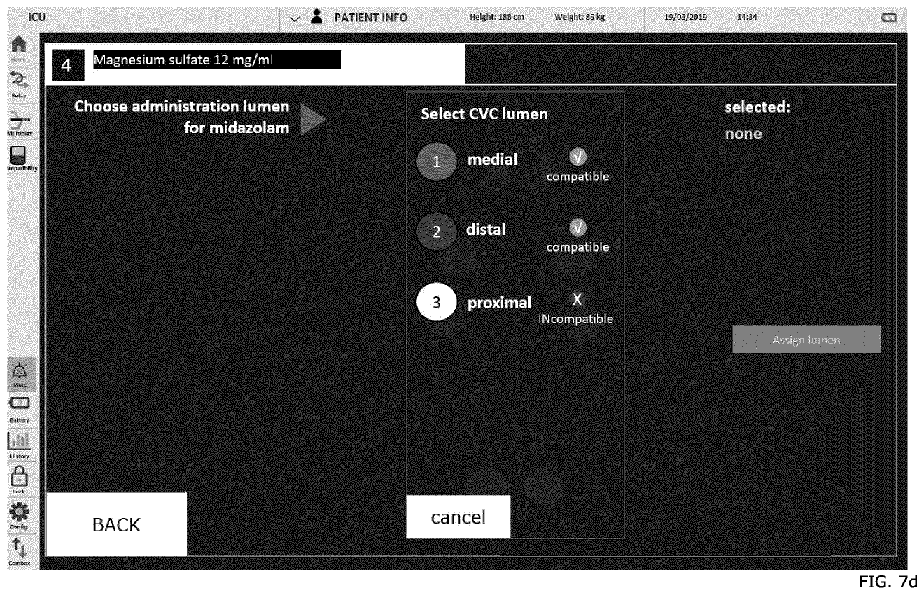

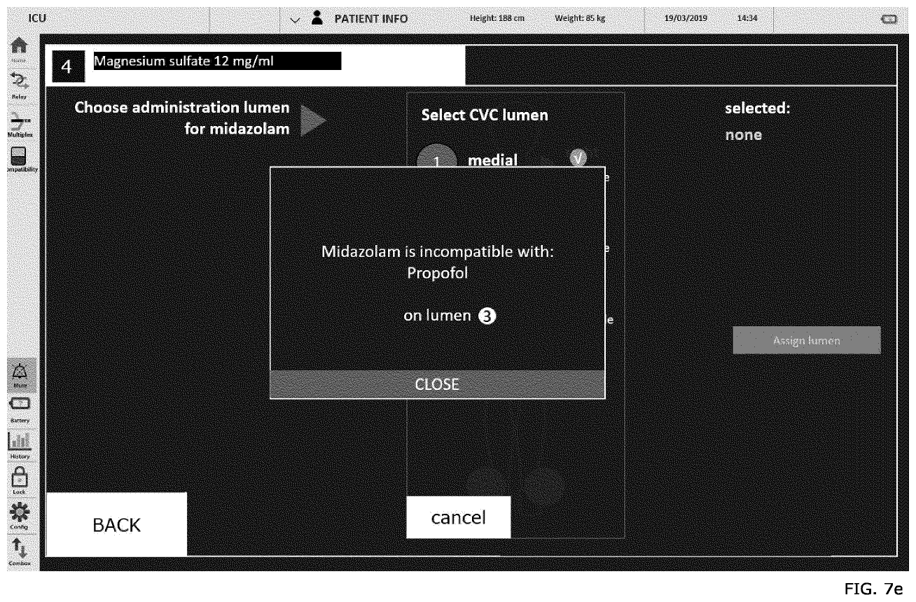

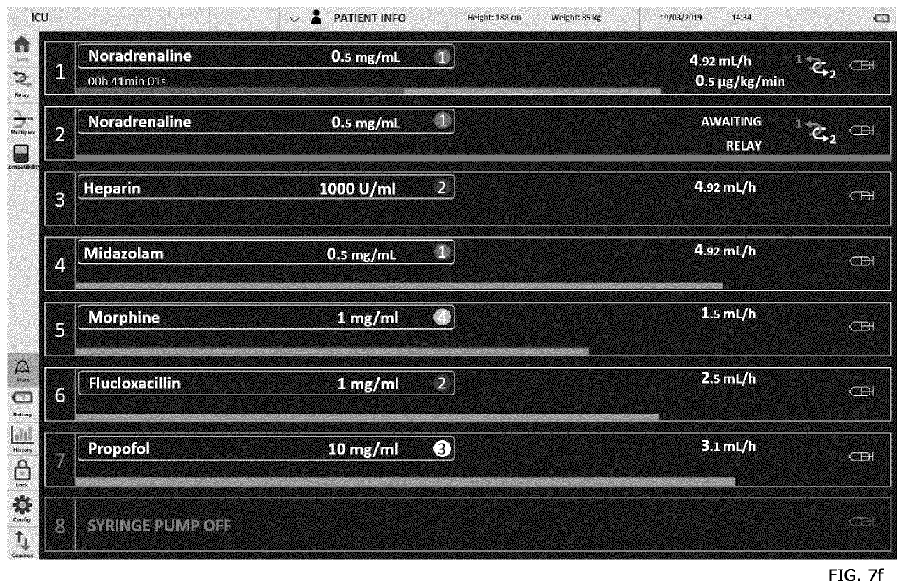

FIGS. 7a to 7f show a fifth workflow of operation related to a lumen advisor with a manual management from a given set of infusion lines;

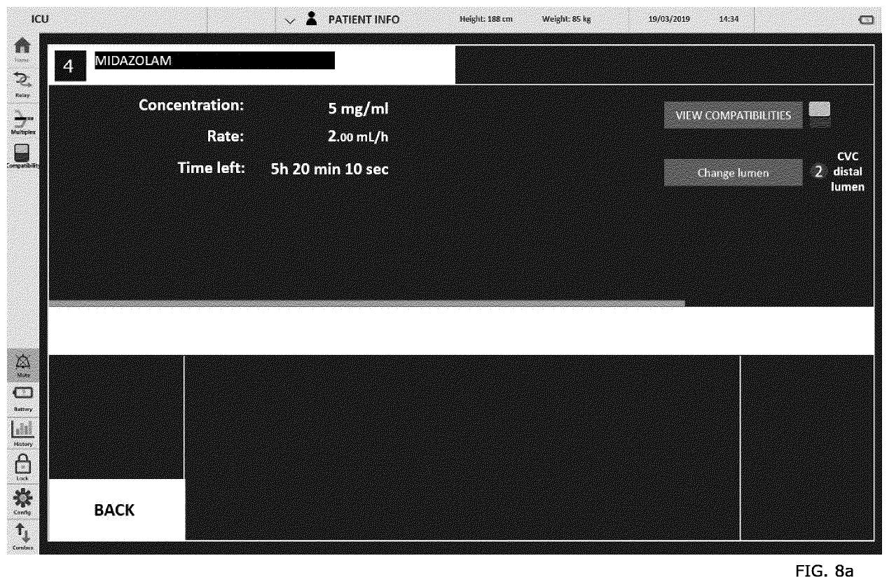

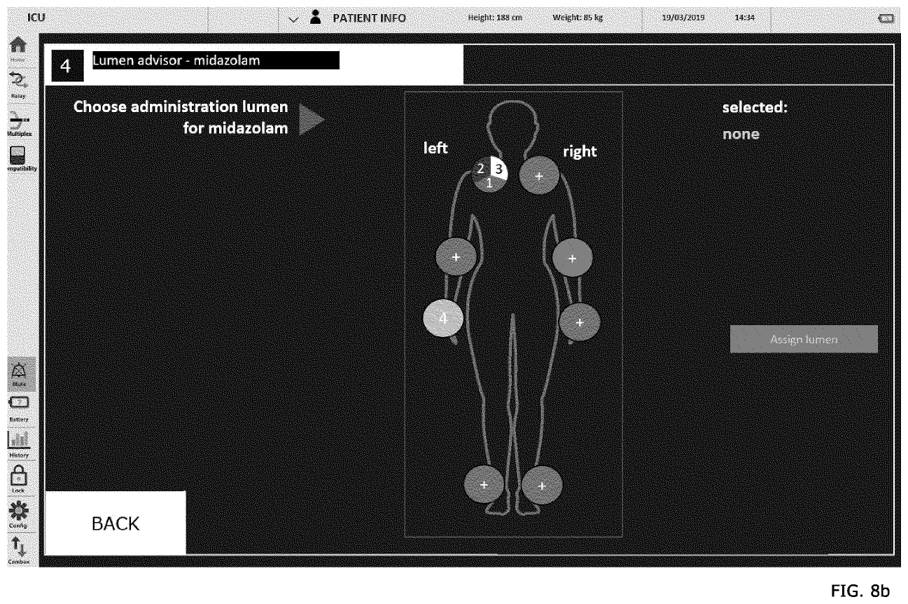

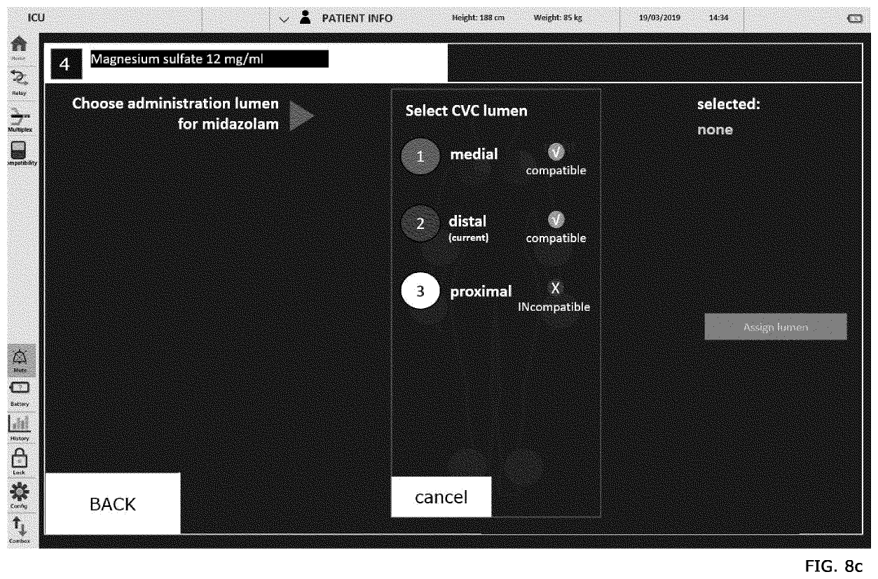

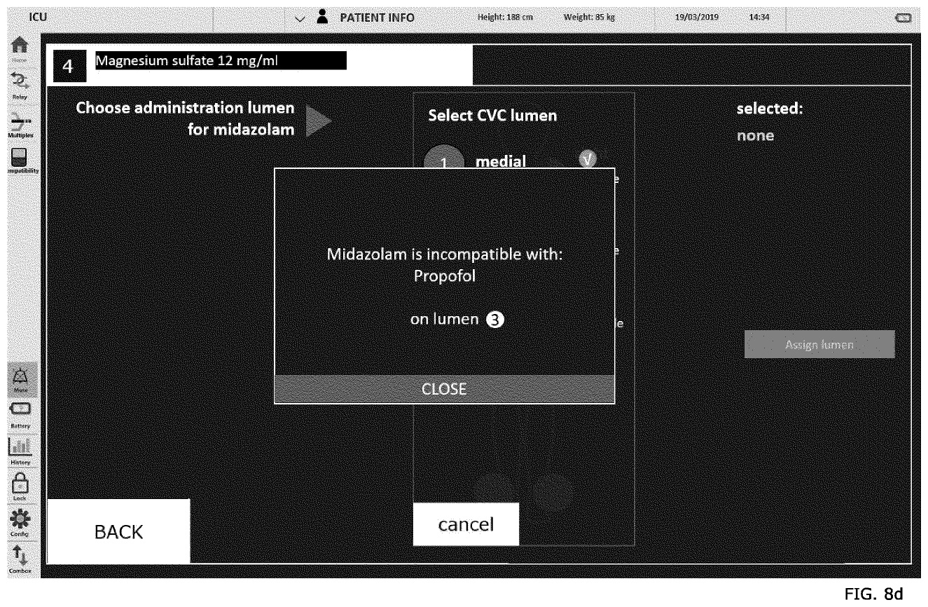

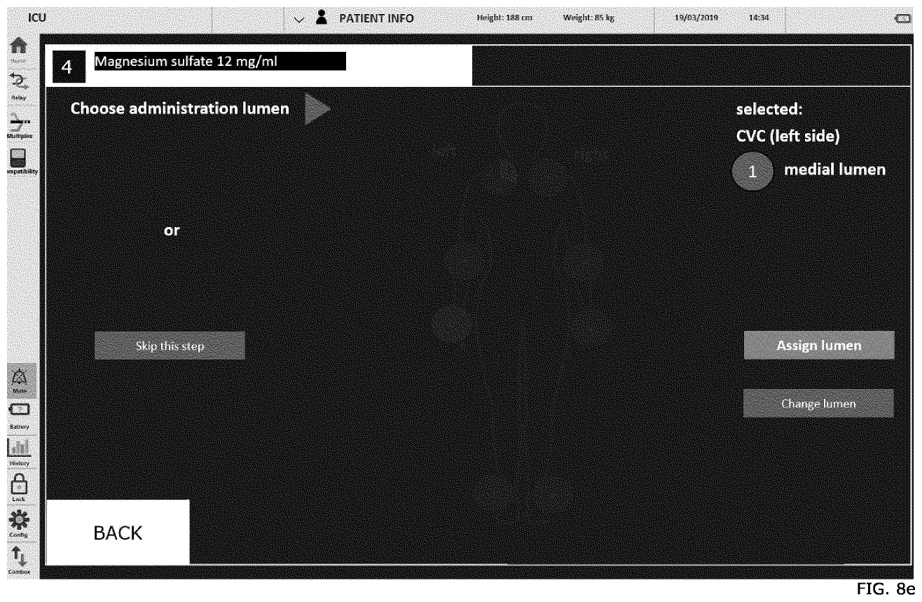

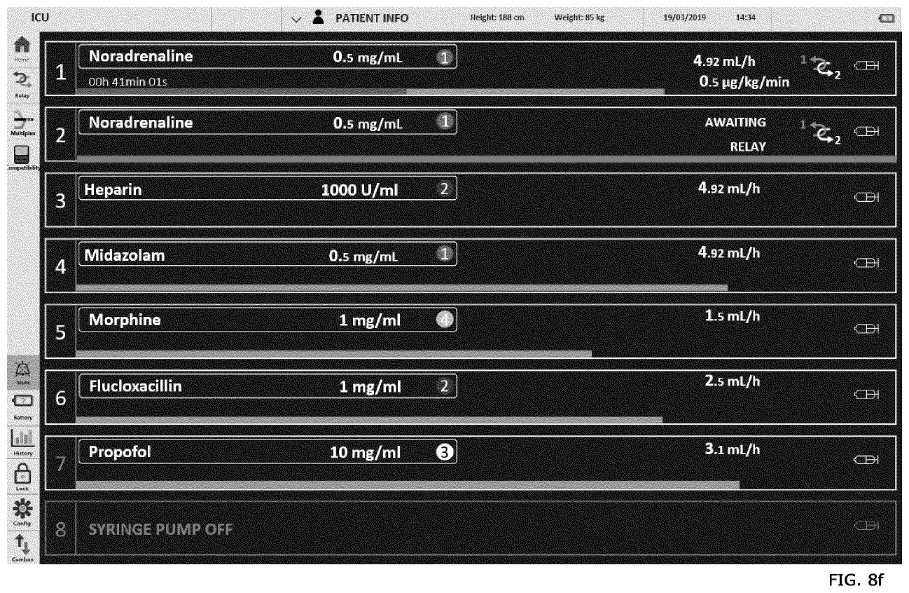

FIGS. 8a to 8f show a sixth workflow of operation related to a lumen advisor with a manual management from a drug detailed menu;

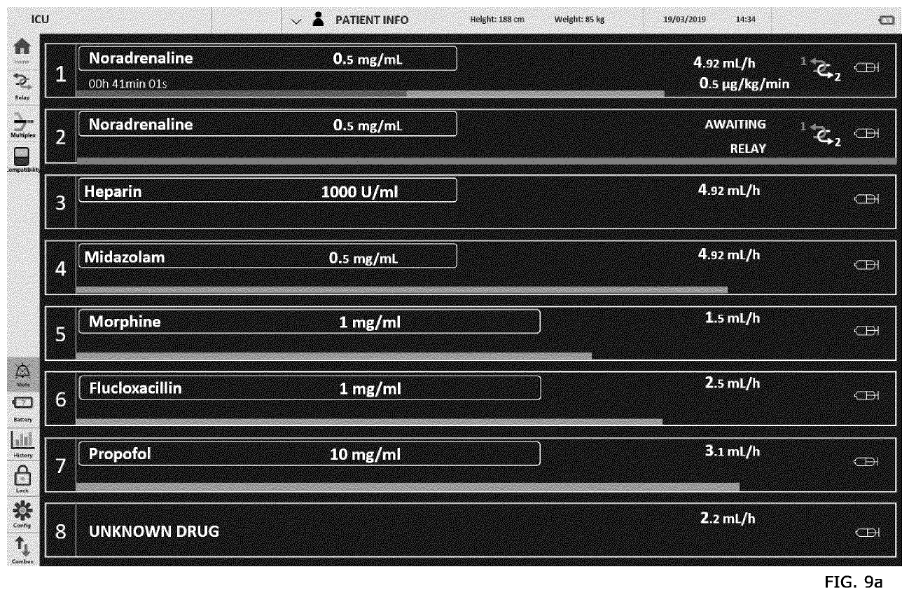

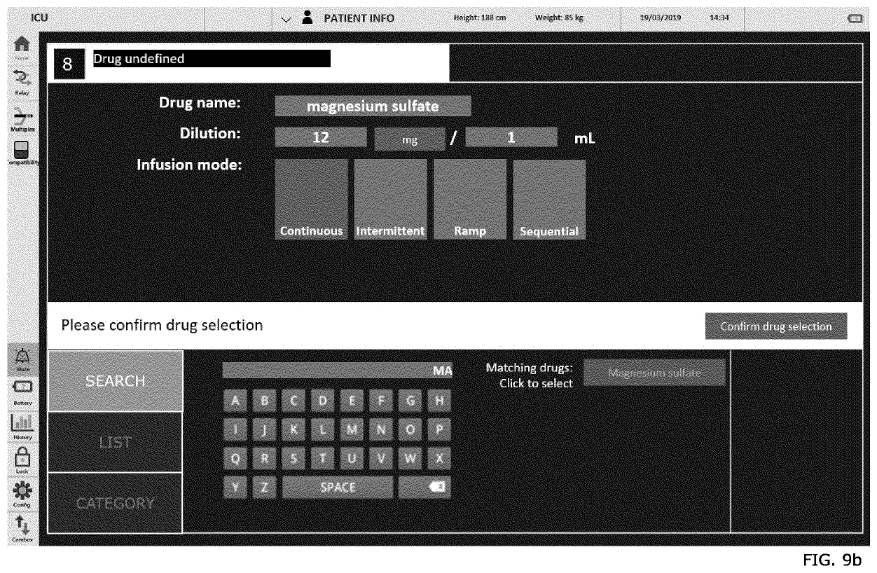

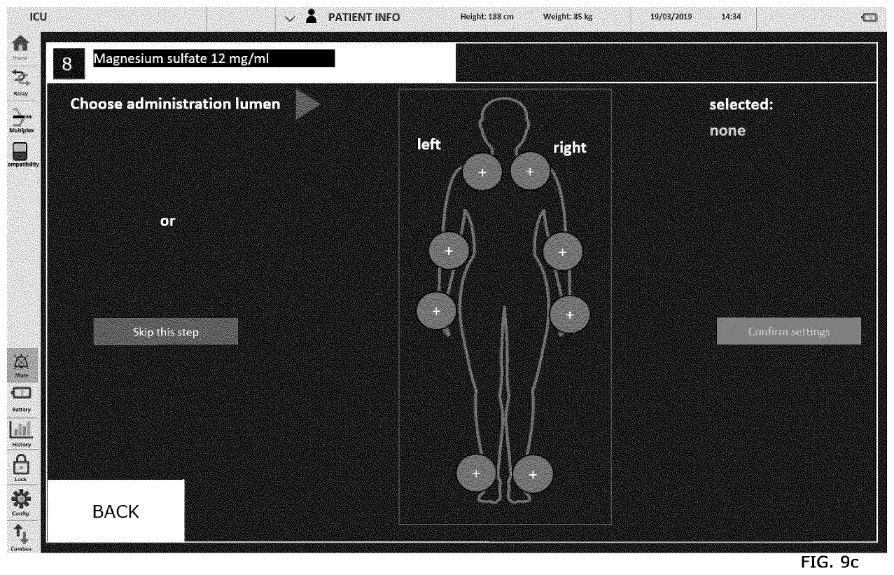

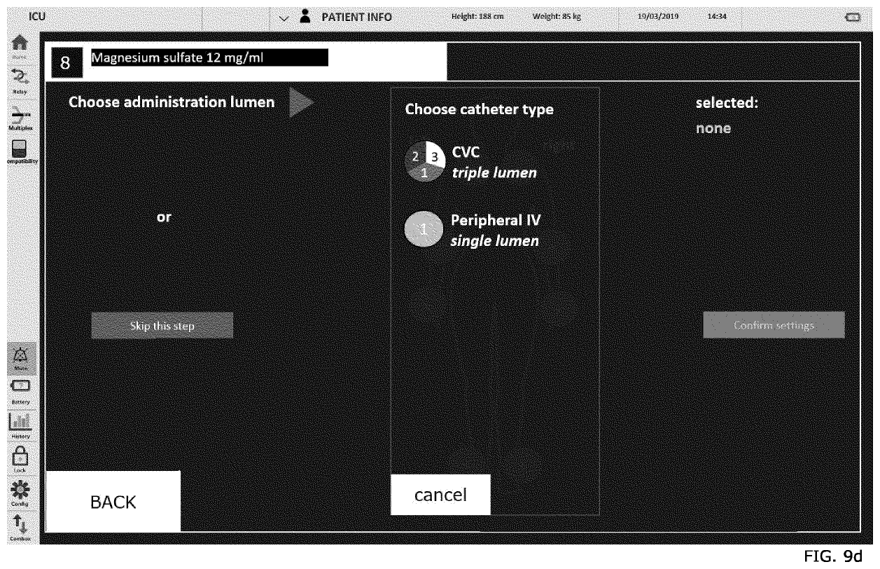

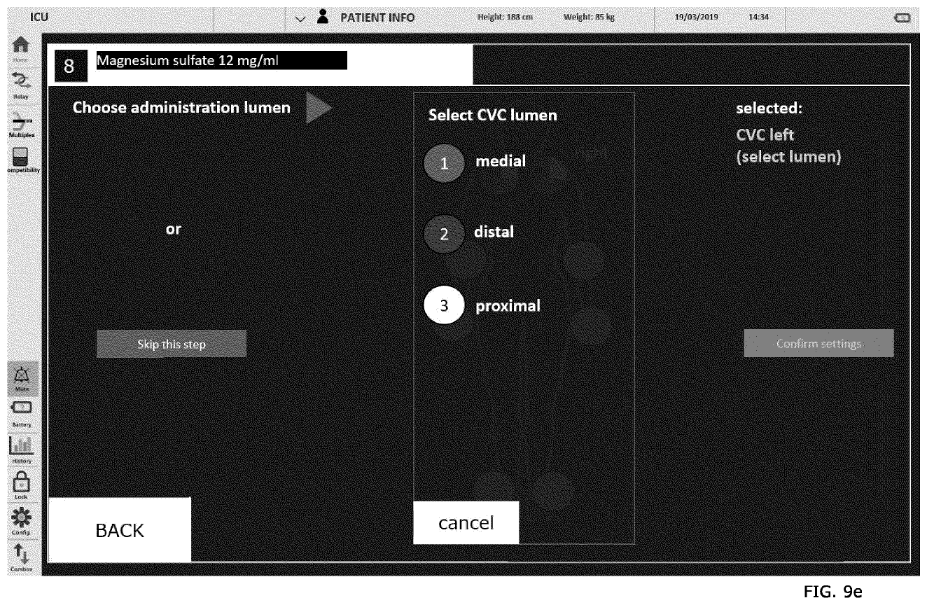

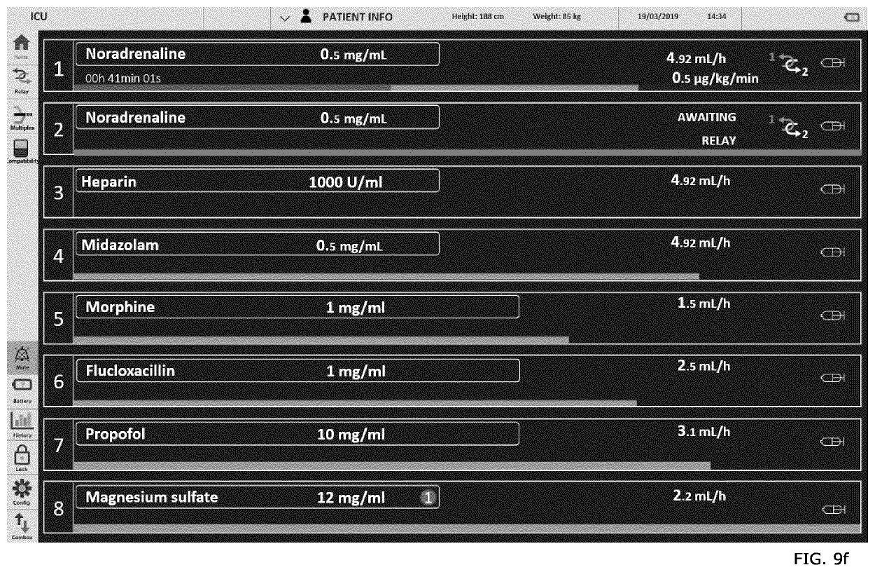

FIGS. 9a to 9f show a seventh workflow of operation related to the creation of a new drug and assignation to a lumen regarding the compatibility;

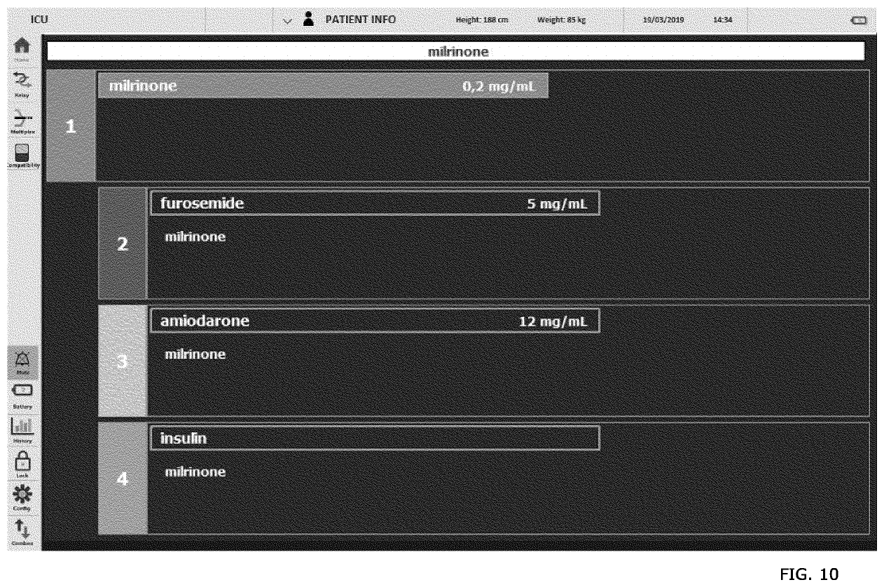

FIGS. 10 to 12 show alternative displays of information regarding the compatibility of infused fluids.

Referring to the schematic view of FIG. 1, in a hospital environment multiple infusion devices 10 may be arranged at the bedside of a patient P, constituting an infusion station 1, for administering multiple medical solutions to the patient P. A scenario of this kind may for example be found in a critical care unit of a hospital, for example an intensive care unit, wherein the orchestrated, coordinated administration of multiple medical solutions to the patient P may be critical to sustain the patient's constitution.

The infusion devices 10, for example in the form of volumetric (peristaltic) or syringe infusion pumps, may for example be arranged on a rack 11, which is constituted to mechanically hold the infusion devices 10 in an organized fashion and to provide a communication backbone in between the infusion devices 10, also connecting the infusion devices 10 to an external communication network, for example a hospital network.

The rack 11 is typically arranged on a stand 12 and in this way is physically placed at the bedside of the patient P.

In the first scenario, the infusion devices 10 are controlled by a control device 2 being linked for example to the rack 11 and via the rack 11 to the infusion devices 10 by means of a communication link 20, for example a wired link or a wireless link. The control device 2 may be implemented for example on a computing device, such as a personal computer, a tablet computer, a laptop computer, a mobile device such as a smart phone, or the like. The control device 2 may be a dedicated physical entity, or alternatively may be implemented by software on a multipurpose computing device.

Within the instant context, the control device 2 controls the infusion devices 10 for an orchestrated infusion of multiple medical solutions, through a given number of delivery lines—catheter and/or tube set—100 and associated disposables like stopcock, filters, . . . .

In relation with FIG. 2, a compatibility chart is provided on a display at the control device 2. It may also be provided at a remote support like a drug library software accessible at a computer or a mobile device by a pharmacist. In a typical use, the pharmacist edits the compatibility chart at the drug library level which then allow the new dataset to be loaded into the infusion devices 2 (via wired or wireless communication) and eventually used by the nurse with the selection and filter function described further on. In both columns and lines headers of this chart, also referred to as a matrix, different names of infusible drugs are displayed. All these drugs may be administrated to the same patient and they typically appear in lines and columns. At the intersection of a given line and a given column, one information is displayed, among three possibilities:
Compatible, means that two considered drugs are compatibles and could be infused at the same time in the same lumen (they could be mixed if needed).
Incompatible, means that drugs are not compatible and cannot be infused in the same lumen nor in the same catheter.
Void (or no information), only refers to two identical drugs being considered and then no compatibility is needed. It can also refer to an unknown compatibility.

The chart content is recorded in a memory of the control device (2). It may also be recorded in a remote server (not shown) and then called by the control device (2) on demand.

The compatibility chart is an editable one, such that the user (typically a pharmacist) is able to modify or even add another drug in the table, upon authorization.

As part of the invention, the control device, prior to displaying the compatibility information to the user, determines all compatibilities and non-compatibilities between infused drugs such that it can provide the user with a comprehensive information allowing a fast and easy decision.

By the term “determines”, it is understood that the control device retrieves compatibility information from an external database or uses memorized compatibility information and then selects the infused or to be infused drugs compatibility information, to be further processed and displayed as it is disclosed further on.

The external database or memorized compatibility information encompasses the comprehensive knowledge of drug compatibility, which can be built and populated whenever a new drug is considered.

Several functions are provided, within the extent of this invention, so as to propose different HMI configurations at the control device (2) level in order to address the need for guidance provided to the user in the management of the preparation of infusing different drugs. All the following described workflows and preferably displayed on the screen are at the control device (2) level, typically selectable by the user. Preferably they are selectable either via a touchscreen ability of the control device (2) or via a dedicated mechanical knob associated to the control device (2), to ease the different operations.

In FIG. 3a to FIG. 3f, a first workflow of operation is provided, in a sequenced fashion, related to the search of compatibility between drugs to be infused, independent of any lumen assignment or infusion.

From a main screen—FIG. 3a—the different drugs to be infused are referred to by dedicated numbered swim lanes associated to different infusing devices. To each of these drugs can be applied and displayed a dose rate as well as an already infused volume (if a previous infusion was performed or if a current infusion is performed), visualized by a progression indicator, like a progress bar. On the left (or as it could be alternatively imagined: on the right or at the bottom or even at the top of the screen) a “compatibility” icon is displayed so that the user is able to enter a sub-menu, visualized on FIG. 3b. This sub-menu is typically displaying options of visualization and operations in relation with drug compatibility management. In this first workflow of operation, the “search compatibility” is selectable so as to enter a list of possible drugs to be infused, depicted in FIG. 3c. This drug list is already known and previously entered into the control device (2) memory or call by the control device (2) from an external server or computer.

From the selection—FIG. 3d—of a first drug, it is provided a visual information—FIG. 3e and FIG. 3f—referring to the compatibility—could be displayed with a tick, for instance green or non-compatibility—could be displayed with a cross, for instance red—with another drug which is then provided as an information to the user. The user is also able to go back into the menus thanks to a dedicated button labeled as “back”. Colors and shape of these ticks can differ from the one presented in this document. In this first workflow, only information is provided to the user.

In FIG. 4a to FIG. 4d, a second workflow is provided, in a sequenced fashion, also related to the search of compatibility between drugs to be infused, but for a given infused or to be infused drug.

From a main screen—FIG. 4a—, similar to the one displayed in FIG. 3a, the user is able to select a given swim lane so as to enter a sub-menu—FIG. 4b—displaying the expected or current dose rate and/or volume infused or to be infused (in the example, swim lane 4 is selected). In this sub-menu, possibilities are given to the user to either view the compatibility list or change the assigned lumen, as it can be appreciated on the right part of the screen view. In this second workflow, the compatibility list is selected so as to enter further screens showing the compatibility, FIG. 4c, or non-compatibility, FIG. 4d, with another drug in a similar manner as the one shown in FIGS. 3e and 3f.

In FIG. 5a to FIG. 5f, a third workflow of operation is shown, in a sequenced fashion, related to the management of the compatibility list. From a first screen, depicted in FIG. 5a, which is similar to the screen viewed in FIG. 3b, the user is able to select the “view compatibility chart” so as to enter the compatibility matrix as shown in FIG. 5b. This compatibility matrix is similar to the matrix viewed on FIG. 2. This compatibility matrix is either used to view a compatibility of a given drug in an easy and intuitive way or used to enter a new drug compatibility information as shown in FIG. 5c and FIG. 5d. It is also possible to select one or several drugs so as to filter and only display their respective compatibilities, as shown in FIG. 5e and FIG. 5f.

FIG. 6a to FIG. 6e show a fourth workflow of operation, in a sequenced fashion, related to user guidance in regard to the lumen assignment of a given drug to be infused. From a first screen, depicted in FIG. 6a and similar to the screen viewed in FIG. 3b and FIG. 5a, the user is able to select a “IC lumen advisor” sub-menu so as to enter a further screen depicted in FIG. 6b. From a list of given drugs to be infused (or already infusing) provided on the left of the screen, it is proposed some possible lumen assignment, defined as “profiles”, depending on the compatibility matrix shown previously. Indeed, the control device (2) either by himself or assisted by an external power computation, is able to calculate and propose different scenarios (profiles) regarding a given number of drugs and a given number of available lumens. Position and type of lumens (CVC, single or multiple lumens) are also taken into account.

FIG. 6b and FIG. 6c, on the right of the screen, the selected profile number is depicted while the corresponding lumen numbers—with corresponding colors—are provided next to the drug list respective to their lumen assignment proposition. The relative position, name and color of the information may vary without departing from the extent of this invention.

FIG. 6b and FIG. 6c show two examples of proposed profiles and lumen assignments which can then be browsed and selected by the user if the proposition is accepted by the user. In the lower part of the screen are provided information to the user like the type and/or the number of lumens to be considered for each profile. Additionally, and optionally, a warning and acknowledgment can be displayed to the user to confirm whenever a selected profile is considered to check if the actual lumens are physically connected. A further visualization screen may be also provided, similar to the one depicted in FIG. 7c so as to help the user in the decision and the verification, as it will be explained further. At the end of this profile selection and acknowledgment, the user may return to the main screen—FIG. 6e—to visualize the swim lanes and to start or to continue the infusion. A lumen number is assigned to each swim lane, in respect to the previous information provided to the user.

In the configuration of the provided screens shown in FIG. 6b or FIG. 6c, the possibility is given to the user to visualize the different available lumen types and their position respective to the body. This visual and intuitive guidance may help the practitioner at the bedside who is able to visually check that the actual lumens are available on the patient.

From FIG. 7a, similar to FIG. 6b or FIG. 6c, the user may enter a sub-menu—FIG. 7b—where he can select a new (or already known) drug to assign to a lumen and then enter a subsequent screen—FIG. 7c—schematically displaying a patient body shape. By selecting a given lumen, a further screen—FIG. 7d—is provided so as to depict the compatibility of the selected drugs, for example in a typical multi-lumen CVC (Central Venous Catheter). The compatibilities are, again, presented in regard to a provided computed information from the compatibility matrix. In this non-limited example, respective to each lumen (for instance distal, medial and proximal ones) the compatibility in respect to the infused drugs is provided. A further warning may be addressed to the user if a non-compatible lumen is wrongly selected, as shown in FIG. 7e. The user is then prompted to enter a compatible one, by selecting a compatible lumen in the screen of FIG. 7d. Eventually, the control device (2) may show the updated swim lanes with the corresponding lumen numbers, FIG. 7f.

As shown in FIG. 8a to FIG. 8f workflow of operation, in a sequenced fashion, the user may also be able to check and select a compatible lumen also from a drug detail menu displayed in FIG. 8a. The user, when managing one specific drug, may indeed be able to select a corresponding lumen—FIG. 8b to FIG. 8d, in a similar way as the one provided from FIG. 7c to FIG. 7e. Once the lumen selected and acknowledged—FIG. 8e—, the control device (2) may show the updated swim lanes with the corresponding lumen numbers, FIG. 8f.

As a further example of the possibilities given by the compatibility matrix as a guidance to the user in the process of infusing several drugs, a further workflow of operation is shown in FIG. 9A to FIG. 9f, in a sequenced fashion, giving the possibility to create a new drug from the main screen depicted in FIG. 9a. By selecting an empty swim lane, for instance line 8 at the bottom of FIG. 9a, the user may enter successive screens—FIG. 9b to 9e—in order to, respectively, create or retrieve a drug name from a given list into the memory of the control device (2), to select an available lumen at the body shape schematic and confirm the selected entry so as to get back to the main screen, now updated—FIG. 9f—.

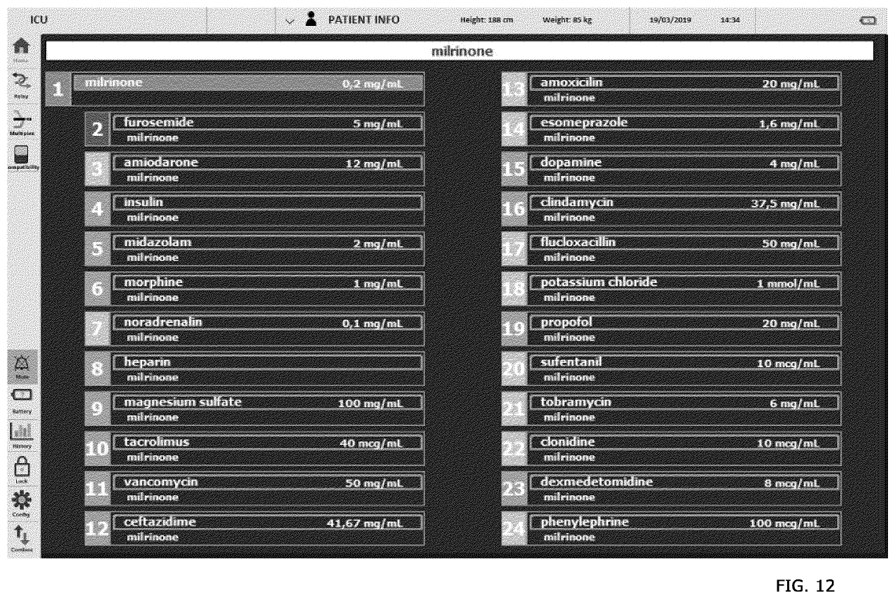

FIGS. 10 to 12 show different views of a same alternative display of compatibility to the user, here typically the nurse. In the first display of FIG. 10, the main screen depicts four different drugs infusing or to be infused through four different infusion devices 2, under stacked lines. It may be preferred, as a non-limitative example, to display the different lines to reflect the actual position of the infusion devices on the infusion station one relative to the others. In this first example, the infusion station has four infusion devices while in FIG. 12, a second example displays twenty-four lines corresponding to two infusion stations with twelve infusion devices being stacked on each one of the stations. In the first example of this FIG. 10, the user selects the first line “milrinone”. The selection information is provided to the user by shifting the row/line to the left and by applying a given color or visual pattern (for example a neutral color like blue or gray). Respective to the selected drug, it is proposed to give information under numbered colored boxes “2”, “3”, “4”, the color depending on the compatibility or non-compatibility of the corresponding drug on the line with the selected drug. For example, a green color indicates a compatibility while a red color indicates a non-compatibility and yellow an unknown compatibility. Of course, different visual information may be envisaged like greyscale of colors, dynamic effects (blinking lines for non-compatibilities) in order to provide the information to the user.

For example, in FIG. 10, “furosemide” is displayed with a dark or red color, since not compatible with “milrinone”, while “insulin” is displayed with a green or lighter color, since compatible with “milrinone”. “Amiodarone” is displayed with a yellow or even brighter color, if compatibility is unknown.

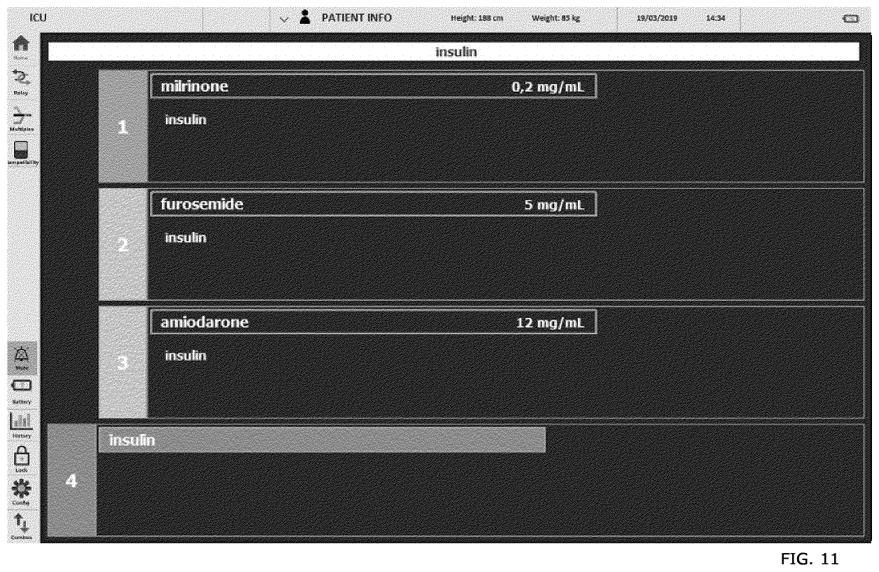

In FIG. 11, with the same four drugs and corresponding lines being considered, but with “insulin” being selected—instead of “milrinone”—, the information provided is adapted such that “milrinone” is displayed with a green color or lighter color—since compatible with “insulin”—while “furosemide” and “amiodarone” are displayed with a yellow or even brighter color since their compatibility is unknown.

In FIG. 12, with several other drugs and lines being displayed, the exact same visual information can be displayed. The number of drugs and compatibility to be displayed is indeed not limited.

### LIST OF REFERENCE NUMERALS

1 Infusion station
2 Control device
10 Infusion device
11 Rack
12 Stand
20 Communication link
100 Delivery lines
P Patient

1. A system for managing multiple infusions to a patient, the system comprising:
a control device configured to control a multiplicity of infusion devices for administering a multiplicity of medical fluids through at least one infusion line of an infusion set to the patient, the multiplicity of medical fluids to the multiplicity of infusion devices being assigned, wherein:
the control device is configured to determine compatibility and non-compatibility of the assigned multiplicity of medical fluids,
the control device is configured to display to a user compatibility information regarding the compatibility or non-compatibility of the multiplicity of medical fluids, and
at least one of the multiplicity of medical fluids to the at least one infusion line is assigned, by the user in accordance with the compatibility information.

2. The system according to claim 1, wherein the multiplicity of medical fluids comprise at least one medical fluid selected from a group consisting of drugs, lactated ringer's solution, saline solution and dextrose solution.

3. The system according to claim 1, wherein the compatibility information is provided as a compatibility list.

4. The system according to claim 3, wherein the compatibility list is a matrix of columns and lines.

5. The system according to claim 1, wherein the compatibility information is editable and searchable by the user.

6. The system according to claim 1, wherein, upon the selection of one of the multiplicity of medical fluids, information on the compatibility or non-compatibility of the one of the multiplicity of medical fluids with at least one another of the multiplicity of medical fluids is provided at the control device to the user, based on the compatibility information.

7. The system according to claim 1, wherein, upon selection of two incompatible fluids, the information of the non-compatibility is displayed at the control device to the user, based on the compatibility information.

8. The system according to claim 1, wherein the control device is further configured to assist the user with a drug-to-lumen assignation based on the compatibility information by providing guidance to address one or several medical fluids of the multiplicity of medical fluids to one or several lumens for administration.

9. The system according to claim 1, wherein the control device is further configured to display a schematic patient body shape with at least one of a localized single or a multiple-channel lumen entry, which is selectable by the user.

10. The system according to claim 9, wherein, upon selection of the one of a localized single or a multiple-channel lumen, a first medical fluid being already assigned to the one of the localized single or the multiple-channel lumen, and upon the selection of a second medical fluid to be infused, the control device is further configured to provide information on compatibility or non-compatibility of the first medical fluid and second medical fluid based on the compatibility information.

11. The system according to claim 1, wherein:
at least one association of an infusion device and an assigned medical fluid to the infusion device is displayed at the control device,
the at least one association being displayed as a progress bar, and
the control device being further configured to provide the user with an updated view of the at least one association with a corresponding lumen indicator.

12. The system according to claim 11, wherein the lumen indicator is a number.

13. The system according to claim 11, wherein the lumen indicator has a dedicated color depending on a selected lumen.

14. The system according to claim 13, wherein the control device comprises a touchscreen in a wired or wireless connection to the multiplicity of infusion devices, the touchscreen commanding the infusion of the multiplicity of infusion devices.

15. A method for managing multiple infusions to a patient by controlling, using a control device, a multiplicity of infusion devices for administering a multiplicity of medical fluids through at least one infusion line of an infusion set to the patient, the method comprising:
assigning by a user the multiplicity of medical fluids to the multiplicity of infusion devices,
determining, by the control device, compatibility and non-compatibility of the assigned multiplicity of medical fluids,
displaying to the user, at the control device, compatibility information regarding the compatibility or non-compatibility of the multiplicity of medical fluids, and
assigning by the user at least one of the multiplicity of medical fluids to the at least one infusion line in accordance with the compatibility information.
